SEQUENCE LISTING (24 sequences)

SEQ ID NO: 1 - protein, 235 aa, synthetic construct
  REGION 1..235: note = cpEGFP
NVYIKADKQKNGIKANFKIRHNIEDGGVQLAYHYQQNTPIGDGPVLLPDNHYLSVQSKLSKDPNEKRDHMVLLEFVTAAGITLGMDELYKMVSKGEELFTGVVPILVELDGDVNGHKFSVSGEGEGDATYGKLTLKFICTTGKLPVPWPTLVTTLTYGVQCFSRYPDHMKQHDFFKSAMPEGYIQERTIFFKDDGNYKTRAEVKFEGDTLVNRIELKGIDFKEDGNILGHKLEYN

SEQ ID NO: 2 - protein, 90 aa, synthetic construct
  REGION 1..90: note = cpEGFP piece 1
NVYIKADKQKNGIKANFKIRHNIEDGGVQLAYHYQQNTPIGDGPVLLPDNHYLSVQSKLSKDPNEKRDHMVLLEFVTAAGITLGMDELYK

SEQ ID NO: 3 - protein, 145 aa, synthetic construct
  REGION 1..145: note = cpEGFP piece 2
MVSKGEELFTGVVPILVELDGDVNGHKFSVSGEGEGDATYGKLTLKFICTTGKLPVPWPTLVTTLTYGVQCFSRYPDHMKQHDFFKSAMPEGYIQERTIFFKDDGNYKTRAEVKFEGDTLVNRIELKGIDFKEDGNILGHKLEYN

SEQ ID NO: 4 - protein, 133 aa, synthetic construct
  REGION 1..133: note = Lanmodulin
MAFRLSSAVLLAALVAAPAYAAPTTTTKVDIAAFDPDKDGTIDLKEALAAGSAAFDKLDPDKDGTLDAKELKGRVSEADLKKLDPDNDGTLDKKEYLAAVEAQFKAANPDNDGTIDARELASPAGSALVNLIR

SEQ ID NO: 5 - protein, 147 aa, synthetic construct
  REGION 1..147: note = Lanmodulin/Calmodulin chimera
DQLTEEQIAEFKEAFSLFDPDKDGTIDLKELGTVMRSLGQNPTEAELQDMINEVDPDKDGTLDAKEFLTMMARKGSYRDTEEEIREAFGVFDPDNDGTLDKKELRHVMTNLGEKLTDEEVDEMIREANPDNDGTIDAREFVQMMTAK

SEQ ID NO: 6 - protein, 434 aa, synthetic construct
  REGION 1..434: note = GLAMOUR 1.0
MVDSSRRKWNKTGHAVRAIGRLSSLENVYIKADKQKNGIKANFKIRHNIEDGGVQLAYHYQQNTPIGDGPVLLPDNHYLSVQSKLSKDPNEKRDHMVLLEFVTAAGITLGMDELYKGGTGGSMVSKGEELFTGVVPILVELDGDVNGHKFSVSGEGEGDATYGKLTLKFICTTGKLPVPWPTLVTTLTYGVQCFSRYPDHMKQHDFFKSAMPEGYIQERTIFFKDDGNYKTRAEVKFEGDTLVNRIELKGIDFKEDGNILGHKLEYNLPMAFRLSSAVLLAALVAAPAYAAPTTTTKVDIAAFDPDKDGTIDLKEALAAGSAAFDKLDPDKDGTLDAKELKGRVSEADLKKLDPDNDGTLDKKEYLAAVEAQFKAANPDNDGTIDARELASPAGSALVNLIRKGELNSKLEGKPIPNPLLGLDSTRTGHHHHHH

SEQ ID NO: 7 - protein, 446 aa, synthetic construct
  REGION 1..446: note = GLAMOUR 2.2
MVDSSRRKWNKTGHAVRAIGRLSSLENVYIKADKQKNGIKANFKIRHNIEDGGVQLAYHYQQNTPIGDGPVLLPDNHYLSVQSKLSKDPNEKRDHMVLLEFVTAAGITLGMDELYKGGTGGSMVSKGEELFTGVVPILVELDGDVNGHKFSVSGEGEGDATYGKLTLKFICTTGKLPVPWPTLVTTLTYGVQCFSRYPDHMKQHDFFKSAMPEGYIQERTIFFKDDGNYKTRAEVKFEGDTLVNRIELKGIDFKEDGNILGHKLEYNLPDQLTEEQIAEFKEAFSLFDKDGTIDLKELGTVMRSLGQNPTEAELQDMINEVDPDKDGTLDAKEFLTMMARKGSYRDTEEEIREAFGVFDPDNDGTLDKKELRHVMTNLGEKLTDEEVDEMIREANPDNDGTIDAREFVQMMTAKKGELNSKLEGKPIPNPLLGLDSTRTGHHHHHH

SEQ ID NO: 8 - DNA, 1302 bp, synthetic construct
  misc_feature 1..1302: note = GLAMOUR 1.0
  source 1..1302: mol_type = other DNA
atggtcgactcatcacgtcgtaagtggaataagacaggtcacgcagtcagagctataggtcggctgagctcactcgagaacgtctatatcaaggccgacaagcagaagaacggcatcaaggcgaacttcaagatccgccacaacatcgaggacggcggcgtgcagctcgcctaccactaccagcagaacacccccatcggcgacggccccgtgctgctgcccgacaaccactacctgagcgtgcagtccaaactttcgaaagaccccaacgagaagcgcgatcacatggtcctgctggagttcgtgaccgccgccgggatcactctcggcatggacgagctgtacaagggcggtaccggagggagcatggtgagcaagggcgaggagctgttcaccggggtggtgcccatcctggtcgagctggacggcgacgtaaacggccacaagttcagcgtgtccggcgagggtgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacatccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaacctgccgatggcgttccgtctgagcagcgcggttctgctggcggcgctggttgcggcgccggcgtatgcggcgccgaccaccaccaccaaggttgacatcgcggcgttcgacccggataaggacggcaccattgacctgaaagaggcgctggcggcgggcagcgcggcgtttgataagctggacccggacaaagatggcaccctggacgcgaaggagctgaaaggccgtgtgagcgaagcggatctgaagaaactggacccggataacgacggcaccctggacaagaaagagtacctggcggcggttgaagcgcagttcaaggcggcgaacccggataacgacggcaccattgatgcgcgtgaactggcgagcccggcgggtagcgcgctggtgaacctgattcgtaagggcgagctcaattcgaagcttgaaggtaagcctatccctaaccctctcctcggtctcgattctacgcgtaccggtcatcatcaccatcaccat

SEQ ID NO: 9 - DNA, 1338 bp, synthetic construct
  misc_feature 1..1338: note = GLAMOUR 2.2
  source 1..1338: mol_type = other DNA
atggtcgactcatcacgtcgtaagtggaataagacaggtcacgcagtcagagctataggtcggctgagctcactcgagaacgtctatatcaaggccgacaagcagaagaacggcatcaaggcgaacttcaagatccgccacaacatcgaggacggcggcgtgcagctcgcctaccactaccagcagaacacccccatcggcgacggccccgtgctgctgcccgacaaccactacctgagcgtgcagtccaaactttcgaaagaccccaacgagaagcgcgatcacatggtcctgctggagttcgtgaccgccgccgggatcactctcggcatggacgagctgtacaagggcggtaccggagggagcatggtgagcaagggcgaggagctgttcaccggggtggtgcccatcctggtcgagctggacggcgacgtaaacggccacaagttcagcgtgtccggcgagggtgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacatccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaacctgccggaccaactgactgaagagcagatcgcagaatttaaagaggctttctccctatttgataaggacggcaccattgacctgaaagagctggggacggtgatgcggtctctggggcagaaccccacagaagcagagctgcaggacatgatcaatgaagtagacccggacaaagatggcaccctggacgcgaaggagttcctgacaatgatggcaagaaaagggagctacagggacacggaagaagaaattagagaagcgttcggtgtgtttgacccggataacgacggcaccctggacaagaaagagcttcgccacgtgatgacaaaccttggagagaagttaacagatgaagaggttgatgaaatgatcagggaagcaaacccggataacgacggcaccattgatgcgcgtgaatttgtacaaatgatgacagcgaagaagggcgagctcaattcgaagcttgaaggtaagcctatccctaaccctctcctcggtctcgattctacgcgtaccggtcatcatcaccatcaccat

SEQ ID NO: 10 - protein, 448 aa, synthetic construct
  REGION 1..448: note = GLamouR 2.3
MVDSSRRKWNKTGHAVRAIGRLSSLENVYIKADKQKNGIKANFKIRHNIEDGGVQLAYHYQQNTPIGDGPVLLPDNHYLSVQSKLSKDPNEKRDHMVLLEFVTAAGITLGMDELYKGGTGGSMVSKGEELFTGVVPILVELDGDVNGHKFSVSGEGEGDATYGKLTLKFICTTGKLPVPWPTLVTTLTYGVQCFSRYPDHMKQHDFFKSAMPEGYIQERTIFFKDDGNYKTRAEVKFEGDTLVNRIELKGIDFKEDGNILGHKLEYNLPDQLTEEQIAEFKEAFSLFDPDKDGTIDLKELGTVMRSLGQNPTEAELQDMINEVDPDKDGTLDAKEFLTMMARKGSYRDTEEEIREAFGVFDPDNDGTLDKKELRHVMTNLGEKLTDEEVDEMIREANPDNDGTIDAREFVQMMTAKKGELNSKLEGKPIPNPLLGLDSTRTGHHHHHH

SEQ ID NO: 11 - protein, 448 aa, synthetic construct
  REGION 1..448: note = GLAMOUR 2.4
MVDSSRRKWNKTGHAVRAIGRLSSLENVYIKADKQKNGIKANFKIRHNIEDGGVQLAYHYQQNTPIGDGPVLLPDNHYLSVQSKLSKDPNEKRDHMVLLEFVTAAGITLGMDELYKGGTGGSMVSKGEELFTGVVPILVELDGDVNGHKFSVSGEGEGDATYGKLTLKFICTTGKLPVPWPTLVTTLTYGVQCFSRYPDHMKQHDFFKSAMPEGYIQERTIFFKDDGNYKTRAEVKFEGDTLVNRIELKGIDFKEDGNILGHKLEYNLPDQLTEEQIAEFKEAFSLFDPDNDRTLDKKELGTVMRSLGQNPTEAELQDMINEVDPDKDGTLDAKEFLTMMARKGSYRDTEEEIREAFGVFDPDNDGTLDKKELRHVMTNLGEKLTDEEVDEMIREADPDKDGTLDAKEFVQMMTAKKGELNSKLEGKPIPNPLLGLDSTRTGHHHHHH

SEQ ID NO: 12 - protein, 449 aa, synthetic construct
  REGION 1..449: note = GLAMOUR-rs
MVDSSRRKWNKAGHAVRAIGRLSSPVVSERMYPEDGALKSEIKKGLRLKDGGHYAAEVKTTYKAKKPVQLPGAYIVDIKLDIVSHNEDYTIVEQCERAEGRHSTGGMDELYKGGTGGSLVSKGEEDNMAIIKEFMRFKVHMEGSVNGHEFEIEGEGEGRPYEAFQTAKLKVTKGGPLPFAWDILSPQFMYGSKAYIKHPADIPDYFKLSFPEGFRWERVMNFEDGGIIHVNQDSSLQDGVFIYKVKLRGTNFPPDGPVMQKKTMGWEATRDDLTEEQIAEFKEAFSLFDPDKDGTIDLKELGTVFRSLGQNPTEAELQDMINEVDPDKDGTLDAKEFLTMMARKMNDTDSEEEIREAFRVFDPDNDGTLDKKELRHVMTDLGEKLTDEEVDEMIRVANPDNDGTIDAREFVQMMTAKKGELNSKLEGKPIPNPLLGLDSTRTGHHHHHH

SEQ ID NO: 13 - protein, 448 aa, synthetic construct
  REGION 1..448: note = GLAMOUR-X
MTRRKKTFKEVATAVKIIAMLMGLKINVYIKADKQKNGIKANFHIRHNIEDGGVQLAYHYQQNTPIGDGPVLLPDNHYLSVESKLSKDPNEKRDHMVLLEFVTAAGITLGMDELYKGGTGGSMVSKGEELFTGVVPILVELDGDVNGHKFSVSGEGEGDATYGKLTLKFICTTGKLPVPWPTLVTTLTYGVQCFSRYPDHMKQHDFFKSAMPEGYIQERTIFFKDDGNYKTRAEVKFEGDTLVNRIELKGIDFKEDGNILGHKLEYNLPDQLTEEQIAEYKEAFSLFDPDKDGTIDLKELGTVMRSLGHNPTEAELQDMINEVDPDKDGTLDAKEFLTMMARKMKYRDTEEEIREAFGVFDPDNDGTLDKKELRHVMTNLGEKLTDEEVDEMIREANPDNDGTIDAREFVQMMTAKKGELNSKLEGKPIPNPLLGLDSTRTGHHHHHH

SEQ ID NO: 14 - protein, 241 aa, synthetic construct
  REGION 1..241: note = cpEGFP
NVYIKADKQKNGIKANFKIRHNIEDGGVQLAYHYQQNTPIGDGPVLLPDNHYLSVQSKLSKDPNEKRDHMVLLEFVTAAGITLGMDELYKGGTGGSMVSKGEELFTGVVPILVELDGDVNGHKFSVSGEGEGDATYGKLTLKFICTTGKLPVPWPTLVTTLTYGVQCFSRYPDHMKQHDFFKSAMPEGYIQERTIFFKDDGNYKTRAEVKFEGDTLVNRIELKGIDFKEDGNILGHKLEYN

SEQ ID NO: 15 - protein, 241 aa, synthetic construct
  REGION 1..241: note = cpEGFP
NVYIKADKQKNGIKANFHIRHNIEDGGVQLAYHYQQNTPIGDGPVLLPDNHYLSVESKLSKDPNEKRDHMVLLEFVTAAGITLGMDELYKGGTGGSMVSKGEELFTGVVPILVELDGDVNGHKFSVSGEGEGDATYGKLTLKFICTTGKLPVPWPTLVTTLTYGVQCFSRYPDHMKQHDFFKSAMPEGYIQERTIFFKDDGNYKTRAEVKFEGDTLVNRIELKGIDFKEDGNILGHKLEYN

SEQ ID NO: 16 - protein, 150 aa, synthetic construct
  REGION 1..150: note = RS fluorescent domain
VSERMYPEDGALKSEIKKGLRLKDGGHYAAEVKTTYKAKKPVQLPGAYIVDIKLDIVSHNEDYTIVEQCERAEGRHSTGGMDELYKGGTGGSLVSKGEEDNMAIIKEFMRFKVHMEGSVNGHEFEIEGEGEGRPYEAFQTAKLKVTKGGP

SEQ ID NO: 17 - protein, 147 aa, synthetic construct; identical to SEQ ID NO: 5
  REGION 1..147: note = Lanmodulin/Calmodulin chimera

SEQ ID NO: 18 - protein, 147 aa, synthetic construct
  REGION 1..147: note = Lanmodulin/Calmodulin chimera
DQLTEEQIAEFKEAFSLFDPDNDRTLDKKELGTVMRSLGQNPTEAELQDMINEVDPDKDGTLDAKEFLTMMARKGSYRDTEEEIREAFGVFDPDNDGTLDKKELRHVMTNLGEKLTDEEVDEMIREADPDKDGTLDAKEFVQMMTAK

SEQ ID NO: 19 - protein, 147 aa, synthetic construct
  REGION 1..147: note = Lanmodulin/Calmodulin chimera
DQLTEEQIAEYKEAFSLFDPDKDGTIDLKELGTVMRSLGHNPTEAELQDMINEVDPDKDGTLDAKEFLTMMARKMKYRDTEEEIREAFGVFDPDNDGTLDKKELRHVMTNLGEKLTDEEVDEMIREANPDNDGTIDAREFVQMMTAK

SEQ ID NO: 20 - protein, 147 aa, synthetic construct
  REGION 1..147: note = Lanmodulin/Calmodulin chimera
DDLTEEQIAEFKEAFSLFDPDKDGTIDLKELGTVFRSLGQNPTEAELQDMINEVDPDKDGTLDAKEFLTMMARKMNDTDSEEEIREAFRVFDPDNDGTLDKKELRHVMTDLGEKLTDEEVDEMIRVANPDNDGTIDAREFVQMMTAK

SEQ ID NO: 21 - DNA, 1344 bp, synthetic construct
  misc_feature 1..1344: note = GLamouR 2.3
  source 1..1344: mol_type = other DNA
atggtcgactcatcacgtcgtaagtggaataagacaggtcacgcagtcagagctataggtcggctgagctcactcgagaacgtctatatcaaggccgacaagcagaagaacggcatcaaggcgaacttcaagatccgccacaacatcgaggacggcggcgtgcagctcgcctaccactaccagcagaacacccccatcggcgacggccccgtgctgctgcccgacaaccactacctgagcgtgcagtccaaactttcgaaagaccccaacgagaagcgcgatcacatggtcctgctggagttcgtgaccgccgccgggatcactctcggcatggacgagctgtacaagggcggtaccggagggagcatggtgagcaagggcgaggagctgttcaccggggtggtgcccatcctggtcgagctggacggcgacgtaaacggccacaagttcagcgtgtccggcgagggtgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacatccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaacctgccggaccaactgactgaagagcagatcgcagaatttaaagaggctttctccctatttgacccggataaggacggcaccattgacctgaaagagctggggacggtgatgcggtctctggggcagaaccccacagaagcagagctgcaggacatgatcaatgaagtagacccggacaaagatggcaccctggacgcgaaggagttcctgacaatgatggcaagaaaagggagctacagggacacggaagaagaaattagagaagcgttcggtgtgtttgacccggataacgacggcaccctggacaagaaagagcttcgccacgtgatgacaaaccttggagagaagttaacagatgaagaggttgatgaaatgatcagggaagcaaacccggataacgacggcaccattgatgcgcgtgaatttgtacaaatgatgacagcgaagaagggcgagctcaattcgaagcttgaaggtaagcctatccctaaccctctcctcggtctcgattctacgcgtaccggtcatcatcaccatcaccat

SEQ ID NO: 22 - DNA, 1344 bp, synthetic construct
  misc_feature 1..1344: note = GLamouR 2.4
  source 1..1344: mol_type = other DNA
atggtggactcctcacgccgcaagtggaataagaccgggcacgcggtccgggcaatcggccgccttagctccttggagaacgtctatatcaaggctgataaacagaagaatggcatcaaagctaacttcaaaattcgccacaacattgaagacggcggcgtccagctcgcgtaccactatcagcaaaatacgccaatcggcgatggtccagtccttcttcctgataatcattacttatcggtacagagtaaattgtcaaaggacccgaacgagaaacgtgatcacatggttctcctcgaattcgttaccgcagcaggtattaccttggggatggatgaattgtataagggcggtacaggtggctctatggtatcaaaaggtgaggaactgttcacgggcgtggtgcctattctcgttgagcttgatggtgatgtgaatggccataaattctcggtatctggcgaaggtgagggcgacgccacgtacgggaaactcaccttgaaatttatttgtacgaccggcaagctcccggtaccttggccgaccctggttacaaccctcacatacggggtccaatgcttcagtcgctacccggaccacatgaagcaacacgatttcttcaaatctgcaatgccggaaggctatattcaggaacgtacaatttttttcaaagacgacggcaattacaaaacccgcgccgaggtaaaatttgaaggtgacaccttagttaatcgtatcgagctgaagggcatcgatttcaaggaggacggcaacattttggggcacaaactggaatataacctcccggaccaactgaccgaggagcaaattgccgagtttaaagaagcattctccctgtttgatccggataatgaccgcaccctggataagaaagagttagggacagtaatgcgctccctgggccagaacccaacagaggctgaattacaggacatgattaacgaagttgatccagataaggacggtacccttgacgcaaaagaatttttgactatgatggcgcgcaaaggttcttatcgtgatacagaggaagaaatccgcgaggcatttggggttttcgacccggacaacgacgggaccctcgacaagaaagaattacgtcatgttatgacaaacctgggggaaaaactcacagacgaggaagttgacgagatgattcgtgaagctgacccggacaaggatgggacactggacgctaaggaattcgtccaaatgatgacggccaagaagggcgagctcaattcgaagcttgaaggtaagcctatccctaaccctctcctcggtctcgattctacgcgtaccggtcatcatcaccatcaccat

SEQ ID NO: 23 - DNA, 1347 bp, synthetic construct
  misc_feature 1..1347: note = GLamourR-rs
  source 1..1347: mol_type = other DNA
atggtcgactcatcacgtcgtaagtggaataaggcaggtcacgcagtcagagctataggtcggctgagctcacccgtggtttccgagcggatgtaccccgaggacggcgccctgaagagcgagatcaagaaggggctgaggctgaaggacggcggccactacgccgccgaggtcaagaccacctacaaggccaagaagcccgtgcagctgcccggcgcctacatcgtggacatcaagttggacatcgtgtcccacaacgaggactacaccatcgtggaacagtgcgaacgcgccgagggccgccactccaccggcggcatggacgagctgtacaagggaggtacaggcgggagtctggtgagcaagggcgaggaggataacatggccatcatcaaggagttcatgcgcttcaaggtgcacatggagggctccgtgaacggccacgagttcgagatcgagggcgagggcgagggccgcccctacgaggcctttcagaccgctaagctgaaggtgaccaagggtggccccctgcccttcgcctgggacatcctgtcccctcagttcatgtacggctccaaggcctacattaagcacccagccgacatccccgactacttcaagctgtccttccccgagggcttcaggtgggagcgcgtgatgaacttcgaggacggcggcattattcacgtgaaccaggactcctccctgcaggacggcgtattcatctacaaggtgaagctgcgcggcaccaacttcccccccgacggccccgtaatgcagaagaagaccatgggctgggaggctacgcgtgacgacctgactgaagagcagatcgcagaatttaaagaggctttctccctatttgacccggataaggacggcaccattgacctgaaagagcttgggacggtgttccgatcgctggggcagaaccccacagaagcagagctgcaggacatgatcaatgaagtagacccggacaaagatggcaccctggacgcgaaggagttcctgacgatgatggcaagaaaaatgaatgacacagacagtgaagaggaaattcgcgaagcgttccgcgtgtttgacccggataacgacggcaccctggacaagaaagagcttcgccacgtgatgacagaccttggagagaagttaacagatgaggaggttgatgaaatgatcagggtagcaaacccggataacgacggcaccattgatgcgcgtgaatttgtacaaatgatgacagcgaagaagggcgagctcaattcgaagcttgaaggtaagcctatccctaaccctctcctcggtctcgattctacgcgtaccggtcatcatcaccatcaccat

SEQ ID NO: 24 - DNA, 1344 bp, synthetic construct
  misc_feature 1..1344: note = GLamourR-X
  source 1..1344: mol_type = other DNA
atgacccggcgtaagaaaacatttaaggaggtggctacggctgttaaaatcattgctatgttaatgggccttaagatcaacgtatacatcaaggcagacaaacaaaaaaacggtattaaagctaattttcatattcgccataacattgaagacgggggtgtccaactcgcgtaccactatcagcagaacacccctatcggtgacggcccggttctgcttccagacaatcactacttaagtgttgaatccaaacttagcaaagacccaaacgaaaaacgtgatcacatggttctgctcgagttcgtgactgcggcggggatcacactcggtatggatgaattatacaaaggtggtaccgggggtagtatggtttccaaaggtgaggagttgttcacaggggtagtaccgattctcgtggaattggatggcgacgtaaacgggcataaattctcggtgtcaggcgaaggcgagggcgatgcaacgtatggcaaattaaccttaaaattcatctgtactaccggcaaattgccggtcccatggcctacgttggtgacaacacttacatacggggttcagtgcttctcccgctaccctgatcacatgaaacaacacgacttcttcaagtctgcgatgccggagggctatatccaagaacgtactattttcttcaaggatgacggcaactataaaacgcgcgcggaggtcaagttcgagggtgataccttggttaaccggattgagttgaaagggattgactttaaggaagacggcaacattcttggccataaattggagtacaatctcccagaccagctgactgaagagcagattgccgaatataaagaggcgttttccttattcgatccagacaaagatggcactattgaccttaaggagcttggcaccgtcatgcgtagtcttggccataatcctacagaagcggagttgcaggacatgattaacgaggtggacccggataaggatgggacactggatgcaaaagaattcttgaccatgatggcacgtaaaatgaaatatcgggatacagaggaggagattcgtgaagcattcggggtcttcgaccctgataatgatggtacgctggacaagaaggagctgcgccacgtgatgaccaaccttggtgagaaactgacggatgaggaagtggacgagatgatccgcgaggcaaatccggacaacgatggtacgatcgacgctcgggagttcgtgcagatgatgacagcgaagaagggcgagctcaattcgaagcttgaaggtaagcctatccctaaccctctcctcggtctcgattctacgcgtaccggtcatcatcaccatcaccat

(19) United States
(12) Patent Application Publication
(10) Pub. No.: US 2024/0183787 A1
(43) Pub. Date: Jun. 6, 2024

(54) LANMODULIN-BASED PROTEIN

(71) Applicant: Board of Trustees of Michigan State University, East Lansing, MI (US)

(72) Inventors: Harvey LEE, Okemos, MI (US); Connor GRADY, Okemos, MI (US); Assaf GILAD, Okemos, MI (US)

(73) Assignee: Board of Trustees of Michigan State University, East Lansing, MI (US)

(21) Appl. No.: 18/411,378

(22) Filed: Jan. 12, 2024

Related U.S. Application Data
(63) Continuation of application No. PCT/US22/75681, filed on Aug. 30, 2022.
(60) Provisional application No. 63/238,495, filed on Aug. 30, 2021.

(51) Int. Cl. G01N 21/77; C07K 14/00; G01N 31/22; G01N 33/24

(52) U.S. Cl. CPC G01N 21/77; C07K 14/00; G01N 31/22; G01N 33/24; C07K 2319/60; G01N 2021/7786

(57) ABSTRACT

Disclosed herein are fusion peptides comprising a fluorescent domain and a rare earth element detection domain capable of binding to a rare earth element and fluorescing when exposed to light when a rare earth element is bound and compositions thereof. The fusion peptide may further comprise a leader domain and a tail domain. The fusion peptide acts as a biosensor to detect and quantify rare earth elements. Methods for imaging internal body structures, detecting rare earth elements in a biosample or environmental sample, and cleaning an environmental site are also presented.

### CROSS-REFERENCE TO RELATED APPLICATIONS

This application is a continuation application of PCT/2022/75681, filed on Aug. 30, 2022, which claims the benefit of U.S. Patent Application No. 63/238,495, filed on Aug. 30, 2021. The entire disclosures of the applications identified in this paragraph are incorporated herein by reference.

### GOVERNMENTAL RIGHTS

This invention was made with government support under NS098231, NS104306, and EB024495 awarded by the National Institutes of Health. The government has certain rights in the invention.

### STATEMENT OF A SEQUENCE LISTING

The present disclosure contains references to amino acid sequences and nucleic acid sequences which have been submitted concurrently herewith as the sequence listing .xml file entitled “000415uscob_SequenceListing.xml,” file size 43,234 bytes, created on Jan. 8, 2024. The aforementioned sequence listing is hereby incorporated by reference in its entirety pursuant to 37 C.F.R. § 1.52(e)(5).

### FIELD

The field of the invention relates to a fusion peptide comprising a fluorescent domain and a rare earth element detection domain that serves as a biosensor for rare earth elements, including gadolinium.

### BACKGROUND

The background description includes information that may be useful in understanding the compositions and methods described herein. It is not an admission that any of the information provided herein is prior art or relevant to the compositions and methods, or that any publication specifically or implicitly referenced is prior art.

Rare earth elements, such as lanthanides, actinides, scandium, and yttrium, are widely used in almost every sector of industry, including in the manufacture of communications equipment, medical equipment and imaging, and permanent magnets. The prevalence of rare earth elements in the modern world has led to the accumulation of rare earth elements in landfills, waste-water, and other sites. Currently, there are no plans to recover rare earth elements from discarded equipment or the environment.

Fluorescing element-detecting proteins have been described previously. For example, GCaMP is a calcium indicator protein comprising an EGFP moiety covalently linked to the calcium-binding messenger protein, calmodulin. GCaMP undergoes a conformational change upon binding to calcium which induces fluorescence following exposure to light at the EGFP's excitation wavelength. See, for example, U.S. Pat. No. 9,518,980, which is incorporated herein by reference. However, the utility of GCaMP is limited in that it is specific for calcium and cannot bind to or detect rare earth elements.

LaMP1 is a lanthanide-detecting biomolecule where lanmodulin is placed between the fluorophores ECFP and citrine. Lanmodulin is capable of binding lanthanides and other rare earth elements. In the absence of a lanthanide, the lanmodulin adopts a three-dimensional structure to separate the ECFP and citrine. In such instances, when ECFP is exposed to light, the molecule fluoresces at ECFP's emission wavelength. However, in the presence of a lanthanide binding induces a conformational change to the lanmodulin that brings the citrine into proximity with the ECFP to induce a FRET response. When light is shown on LaMP1 in the presence of a lanthanide, the molecule fluoresces at citrine's emission wavelength. In such a manner, LaMP1 can act as a sensor for lanthanide and actinide elements. See, for example, WO 2020/051274, which is incorporated herein by reference.

### SUMMARY

A fusion peptide comprising a fluorescent domain and a rare earth element detection domain, and a composition thereof, is disclosed herein. The fusion peptide may further comprise a leader domain and a tail domain. In certain embodiments, the fluorescent domain comprises enhanced green fluorescent protein (EGFP) and the rare earth element detection domain comprises lanmodulin or a chimera of lanmodulin and calmodulin. In certain embodiments, the rare earth element detection domain detects gadolinium.

Also disclosed herein are cells comprising the fusion peptide. Nucleic acids encoding the fusion peptide and vectors comprising the nucleic encoding the fusion peptide are also disclosed.

Also disclosed herein are methods for imaging one or more internal body structures in a subject, the method comprising administering to the subject a contrast agent, administering to the subject a composition comprising the fusion peptide, detecting an amount of fluorescence of the fusion peptide, and preparing an image of the one or more internal body structures based on the location and intensity of the fluorescence.

Also disclosed herein are methods for detecting one or more rare earth elements in a biosample from a subject, the method comprising obtaining the biosample from the subject, contacting the biosample with a composition comprising the fusion peptide, exposing the biosample to light, and determining the amount of fluorescence of the biosample. In certain embodiments, the subject ingested, is suspected of ingesting, or otherwise has been exposed to a rare earth element.

Also described herein are methods for detecting one or more rare earth elements in a sample, such as an environmental sample, the method comprising obtaining the sample, contacting the sample with a composition comprising the fusion peptide, exposing the sample to light, and determining the amount of fluorescence of the sample. In certain embodiments, the sample comprises or is suspected of comprising a rare earth element.

Also described herein are methods for removing one or more rare earth elements from a site containing or suspected of containing the one or more rare earth elements, the method comprising obtaining a sample from the site, contacting the sample with a composition comprising the fusion peptide, incubating the sample for a period of time sufficient for the fusion peptide to bind to one or more rare earth elements, isolating and removing the fusion peptide from the sample, and returning the sample to the site.

Various objects, features, aspects, and advantages will become more apparent from the following detailed description of preferred embodiments, along with the accompanying drawings.

### BRIEF DESCRIPTION OF THE DRAWINGS

FIG. 1 depicts an embodiment of the fusion peptide comprising EGFP piece 1 and EGFP piece 2 as the fluorescent domain, lanmodulin as the rare earth detection domain, M13 as the leader domain, and V5 as the tail domain.

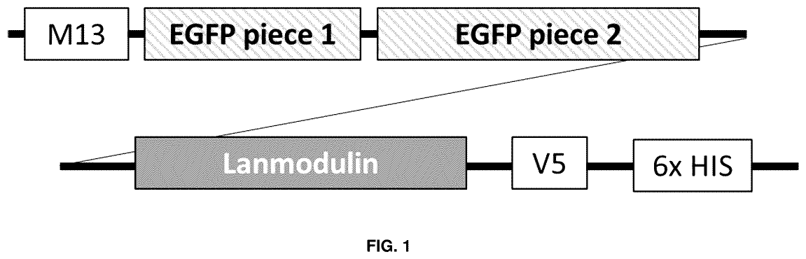

FIG. 2 depicts an embodiment of the fusion peptide comprising EGFP piece 1 and EGFP piece 2 as the fluorescent domain, a chimera of lanmodulin and calmodulin as the rare earth detection domain, M13 as the leader domain, and V5 as the tail domain.

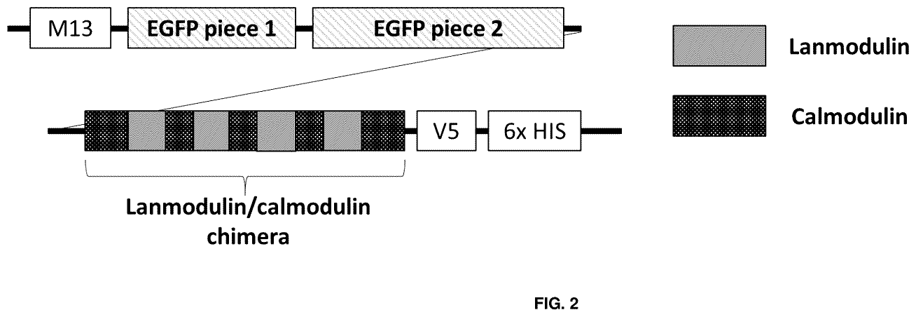

FIG. 3 depicts a proposed mechanism for detecting rare earth elements with the fusion peptide described herein.

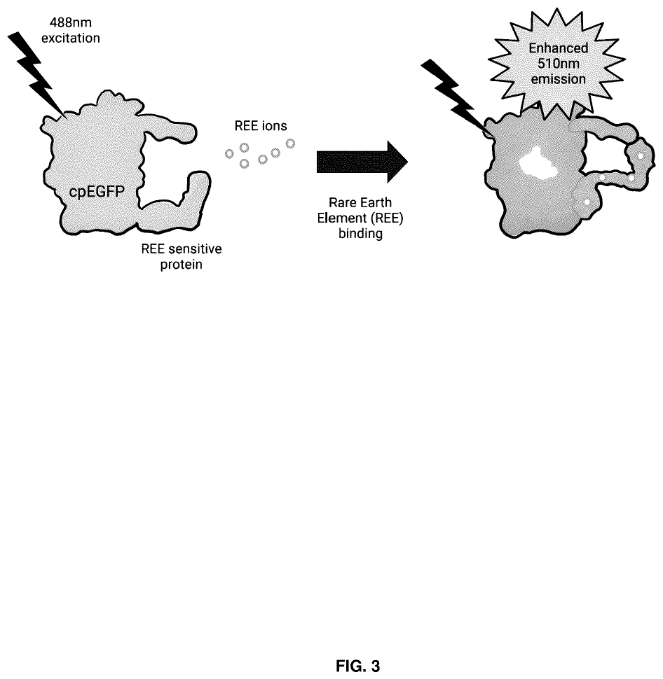

FIG. 4A depicts the change in fluorescence over time of the fusion peptide GLamouR 1.0 following incubation with rare earth elements. FIG. 4B depicts the change in fluorescence over time of the fusion peptide GLamouR 2.2 following incubation with rare earth elements. GLamouR 1.0 represents an embodiment wherein the fusion peptide comprises lanmodulin as the rare earth element detection domain. GLamouR 2.2 represents an embodiment wherein the fusion peptide comprises a chimera of lanmodulin and calmodulin as the rare earth element detection domain.

FIG. 5 depicts the comparison of the fluorescence of SEQ ID NO:7 (GLamouR-2.2) and SEQ ID NO:12 (GLamouR-rs) upon binding of various rare earth elements.

FIG. 6 depicts the fluorescence of SEQ ID NO:7 (GLamouR-2.2) in the absence (grey bars) and presence (hatched bars) of UV exposure.

### DETAILED DESCRIPTION

### Definitions

As used herein, “fusion peptide” or “fusion protein” refers to an amino acid sequence that comprises at least a fluorescent domain and a rare earth element detection domain. The fluorescent domain and the rare earth element detection domain are conjugated to form a single amino acid sequence. The fusion peptide may further comprise a leader domain and a tail domain.

A “fluorescent domain” includes by way of non-limiting examples an amino acid sequence, or a nucleotide sequence that encodes the amino acid sequence, which fluoresces (i.e., emits) a certain wavelength of light when exposed to an excitation wavelength. Fluorescent proteins and their sequences are known in the art and are not limited here. Various fluorescent proteins can be found, for example, at FPbase (www.fbpase.org). In certain embodiments, the fluorescent domain of the fusion peptide described herein may be selected from the group consisting of enhanced green fluorescent protein (EGFP), green fluorescent protein (GFP), enhanced yellow fluorescent protein (EYFP), enhanced blue fluorescent protein (EBFP), enhanced cyan fluorescent protein (ECFP), enhanced red fluorescent protein (ERFP), and enhanced orange fluorescent protein (EOFP). Additionally or alternatively, the fluorescent domain may comprise two or more pieces of a fluorescent protein that are separated by an amino acid sequence that is not with the putative sequence of the fluorescent protein. As a non-limiting example, the fluorescent domain may comprise two pieces of EGFP (e.g., Piece 1 and Piece 2) which are separated by an amino acid sequence that is not within EGFP and, when combined, the two pieces make up the entirety of EGFP. In other embodiments, the fluorescent protein may have at least 70% identity (i.e., at least 75% sequence identity, at least 80% sequence identity, at least 85% sequence identity, at least 90% sequence identity, at least 95% sequence identity, at least 96% sequence identity, at least 97% sequence identity, at least 98% sequence identity, or at least 99% sequence identity) to the putative sequences for EGFP, GFP, EYFP, EBFP, ECFP, ERFP, and/or EOFP.

A “rare earth element detection domain” includes by way of non-limiting examples an amino acid sequence, or a nucleotide sequence that encodes the amino acid sequence, which binds to a rare earth element. In some embodiments, the rare earth element detected by the rare earth element detection domain may bind (e.g., detects) one or more lanthanides, one or more actinides, scandium, yttrium, and combinations thereof. In a particular embodiment, the rare earth element detection domain binds (e.g., detects) one or more of scandium, yttrium, lanthanum, cerium, praseodymium, neodymium, promethium, samarium, europium, gadolinium, terbium, dysprosium, holmium, erbium, thulium, ytterbium, lutetium, thorium, protactinium, uranium, neptunium, plutonium, americium, curium, berkelium, californium, einsteinium, fermium, mendelevium, nobelium, lawrencium, and combinations thereof. Various rare earth element detection domains are known in the art and are not particularly limited so long as the rare earth element detection domain binds to a rare earth element. In some embodiments, the rare earth element detection domain comprises lanmodulin or a chimera of lanmodulin and calmodulin. In certain embodiments, the rare earth element detection domain comprises an amino acid sequence having at least 70% identity (i.e., at least 75% sequence identity, at least 80% sequence identity, at least 85% sequence identity, at least 90% sequence identity, at least 95% sequence identity, at least 96% sequence identity, at least 97% sequence identity, at least 98% sequence identity, or at least 99% sequence identity) to SEQ ID NO:4. In other embodiments, the rare earth element detection domain comprises an amino acid sequence having at least 70% identity (i.e., at least 75% sequence identity, at least 80% sequence identity, at least 85% sequence identity, at least 90% sequence identity, at least 95% sequence identity, at least 96% sequence identity, at least 97% sequence identity, at least 98% sequence identity, or at least 99% sequence identity) to SEQ ID NO:5.

“Chimera” includes by way of a non-limiting example, peptide sequences that combine and/or intermix portions of two or more peptides or proteins. As an example, a chimera of lanmodulin and calmodulin is an amino acid sequence that comprises sequences of lanmodulin interspersed with sequences from calmodulin.

A “leader domain” includes by way of non-limiting examples an amino acid sequence, or a nucleotide sequence that encodes the amino acid sequence, which possesses an affinity to bind the rare earth element detection domain that has itself bound to a rare earth element. The interaction of the leader domain with the rare earth element detection domain bound to a rare earth element occurs through non-covalent means, e.g., through peptide-peptide interactions. Through this binding, the shape of the fusion peptide is changed such that the emission wavelength of the fluorescent domain is enhanced. The leader domain is not particularly limited so long as it maintains an affinity for the rare earth element detection domain bound to a rare earth element. In particular embodiments, the leader sequence may be M13 or RS20, both of which are known in the art. In other embodiments, the leader domain comprises an amino acid sequence having at least 70% identity (i.e., at least 75% sequence identity, at least 80% sequence identity, at least 85% sequence identity, at least 90% sequence identity, at least 95% sequence identity, at least 96% sequence identity, at least 97% sequence identity, at least 98% sequence identity, or at least 99% sequence identity) to M13 or RS20.

A “tail domain” includes by way of non-limiting examples an amino acid sequence, or a nucleotide sequence that encodes the amino acid sequence, which does not interfere with protein folding. In some embodiments, the tail domain acts as a tag to isolate, detect, identify, and/or purify the protein. The isolation, detection, identification, and/or purification can be achieved through known molecular biological techniques, including but not limited to, western blot, flow cytometry, and enzyme immunoassays. Additionally or alternatively, the tail domain enhances solubility of the fusion peptide. Tail domain as a tag is not particularly limited and numerous tags are known in the art, including but not limited to V5, HIS, ALFA, AviTag, C-tag, polyglutamine, polyarginine, E-tag, hemagglutinin, Myc, NE, Rho1D4, S-tag, SBP-tag, Softag, Spot-tag, Strep-tag, T7, TC-tag, Ty-tag, VSV, or Xpress. In other embodiments, the tail domain comprises an amino acid sequence having at least 70% identity (i.e., at least 75% sequence identity, at least 80% sequence identity, at least 85% sequence identity, at least 90% sequence identity, at least 95% sequence identity, at least 96% sequence identity, at least 97% sequence identity, at least 98% sequence identity, or at least 99% sequence identity) to one of V5, HIS, ALFA, AviTag, C-tag, polyglutamine, polyarginine, E-tag, hemagglutinin, Myc, NE, Rho1D4, S-tag, SBP-tag, Softag, Spot-tag, Strep-tag, T7, TC-tag, Ty-tag, VSV, or Xpress.

“Encoding”-when used in reference to a nucleic acid-conveys that when transcription is initiated from the nucleic acid in a cell or an acellular environment, the mRNA transcript produced would be translated into a given protein. In such a manner, the nucleic acid “encodes” a peptide when the codon triplets of tRNA would produce the polypeptide from the nucleic acid according to the ordinary workings of transcription and translation in the cell. In particular embodiments, the nucleic acid encodes the fusion peptide described herein.

“Effective amount” includes by way of non-limiting examples the amount and/or dosage, and/or dosage regime of a composition comprising the fusion peptide described herein necessary to bring about the desired result e.g., an amount sufficient bind to one or more rare earth elements in a sample, biosample, environmental sample, and/or sample from an environmental site.

“Subject,” “individual,” and “patient” interchangeably refer to a mammal, preferably a human or a non-human primate, but also domesticated mammals (e.g., canine or feline), laboratory mammals (e.g., mouse, rat, rabbit, hamster, guinea pig), and agricultural mammals (e.g., equine, bovine, porcine, ovine). In certain embodiments, the subject can be human (e.g., adult male, adult female, adolescent male, adolescent female, male child, female child) under the care of a physician or other health worker. In certain embodiments the subject may not be under the care of a physician or other health worker.

FUSION PEPTIDES AND COMPOSITIONS THEREOF: The fusion peptide described herein relates to an amino acid sequence, or a nucleotide sequence that encodes the amino acid sequence, that comprises a fluorescent domain and a rare earth element detection domain. In particular, the fusion peptide comprises a fluorescent domain conjugated to a rare earth element detection domain. In some embodiments, the fusion peptide further comprises a leader domain and a tail domain. In a specific embodiment the fluorescent domain is conjugated to a leader domain and the rare earth element detection domain is further conjugated to a tail domain.

In an embodiment, the fluorescent domain of the fusion peptide comprises an amino acid sequence having at least 70% identity to a fluorescent protein selected from the group consisting of EGFP, GFP, EYFP, EBFP, ECFP, ERFP, EOFP, and mApple. In a further embodiment, the fluorescent protein is EGFP and the EGFP is circular permutated EGFP (cpEGFP). In a specific embodiment, the fluorescent domain comprises a cpEGFP of SEQ ID NOs:1, 14, or 15. In a specific embodiment, the fluorescent domain comprises a cpEGFP of SEQ ID NOs:2 and 3. In a specific embodiment, the fluorescent domain comprises an mApple of SEQ ID NO: 16.

In an embodiment, the rare earth element detection domain of the fusion peptide comprises an amino acid sequence having at least 70% sequence to a protein selected from the group consisting of lanmodulin and a chimera of lanmodulin and calmodulin. In a specific embodiment, the rare earth element detection domain comprises lanmodulin. In particular the lanmodulin comprises SEQ ID NO:4. In another embodiment, the rare earth element detection domain comprises a chimera of lanmodulin and calmodulin. In particular the lanmodulin/calmodulin chimera comprises one of SEQ ID NOs:5, 17, 18, 19, or 20.

In a specific embodiment, the fusion peptide comprises cpEGFP as the fluorescent domain and lanmodulin as the rare earth element detection domain. In a further embodiment, the fusion peptide further comprises a leader domain having at least 70% sequence identity to M13 or RS20 where the leader domain may be conjugated to the fluorescent domain. In a further embodiment, the fusion peptide also comprises a tail domain, wherein the tail domain comprises an amino acid sequence having at least 70% sequence identity to V5, HIS, ALFA, AviTag, C-tag, polyglutamine, polyarginine, E-tag, hemagglutinin, Myc, NE, Rho1D4, S-tag, SBP-tag, Softag, Spot-tag, Strep-tag, T7, TC-tag, Ty-tag, VSV, or Xpress. In a still further embodiment, the fusion peptide comprises a fluorescent domain comprising SEQ ID NO:1, a rare earth element detection domain comprising SEQ ID NO:4, a leader domain, and a tail domain. In a particular embodiment, the fusion peptide comprises SEQ ID NO:6.

In an alternative embodiment, the fusion peptide comprises cpEGFP as the fluorescent domain and a chimera of lanmodulin and calmodulin as the rare earth element detection domain. In a further embodiment, the fusion peptide further comprises a leader domain having at least 70% sequence identity to M13 or RS20 where the leader domain may be conjugated to the fluorescent domain. In a further embodiment, the fusion peptide also comprises a tail domain, wherein the tail domain comprises an amino acid sequence having at least 70% sequence identity to V5, HIS, ALFA, AviTag, C-tag, polyglutamine, polyarginine, E-tag, hemagglutinin, Myc, NE, Rho1D4, S-tag, SBP-tag, Softag, Spot-tag, Strep-tag, T7, TC-tag, Ty-tag, VSV, or Xpress. In a still further embodiment, the fusion peptide comprises a fluorescent domain comprising SEQ ID NO:1, a rare earth element detection domain comprising SEQ ID NO:5, a leader domain, and a tail domain. In a particular embodiment, the fusion peptide comprises SEQ ID NO:7. In another embodiment, the fusion peptide comprises a fluorescent domain comprising SEQ ID NO:14, a rare earth element detection domain comprising SEQ ID NO:17, a leader domain, and a tail domain. In a particular embodiment, the fusion peptide comprises SEQ ID NO:10. In another embodiment, the fusion peptide comprises a fluorescent domain comprising SEQ ID NO: 14, a rare earth element detection domain comprising SEQ ID NO:18, a leader domain, and a tail domain. In a particular embodiment, the fusion peptide comprises SEQ ID NO:11. In another embodiment, the fusion peptide comprises a fluorescent domain comprising SEQ ID NO: 15, a rare earth element detection domain comprising SEQ ID NO:19, a leader domain, and a tail domain. In a particular embodiment, the fusion peptide comprises SEQ ID NO:13.

In an alternative embodiment, the fusion peptide may be a red-shifted fusion peptide which comprises mApple as the fluorescent domain and a chimera of lanmodulin and calmodulin as the rare earth element detection domain. In a further embodiment, the fusion peptide further comprises a leader domain having at least 70% sequence identity to M13 or RS20 where the leader domain may be conjugated to the fluorescent domain. In a further embodiment, the fusion peptide also comprises a tail domain, wherein the tail domain comprises an amino acid sequence having at least 70% sequence identity to V5, HIS, ALFA, AviTag, C-tag, polyglutamine, polyarginine, E-tag, hemagglutinin, Myc, NE, Rho1D4, S-tag, SBP-tag, Softag, Spot-tag, Strep-tag, T7, TC-tag, Ty-tag, VSV, or Xpress. In a still further embodiment, the fusion peptide comprises a fluorescent domain comprising SEQ ID NO:16, a rare earth element detection domain comprising SEQ ID NO:20, a leader domain, and a tail domain. In a particular embodiment, the fusion peptide comprises SEQ ID NO:12.

Table 1 describes specific combinations of the fluorescent domain, rare earth element detection domain, leader domain, and tail domain.

TABLE 1

| | Domain | | | | SEQ | SEQ |
|---|---|---|---|---|---|---|
| | | | Rare Earth Element | | ID NO | ID NO |
| Name | Leader | Fluorescent | Detection | Tail | (peptide): | (nucleotide): |
| GLamouR | M13 | cpEGFP | lanmodulin | V5 | 6 | 8 |
| 1.0 | | | | | | |
| GLamouR | M13 | cpEGFP | lanmodulin/calmodulin | V5 | 7 | 9 |
| 2.2 | | | chimera | | | |
| GLamouR | M13 | cpEGFP | lanmodulin/calmodulin | V5 | 10 | 21 |
| 2.3 | | | chimera | | | |
| GLamouR | M13 | cpEGFP | lanmodulin/calmodulin | V5 | 11 | 22 |
| 2.4 | | | chimera | | | |
| GLamouR | M13 | mApple | lanmodulin/calmodulin | V5 | 12 | 23 |
| rs | | | chimera | | | |
| GLamouR | M13 | cpEGFP | lanmodulin/calmodulin | V5 | 13 | 24 |
| X | | | chimera | | | |

SEQ ID NOs:6, 7, and 10-13 are offered only as representative examples of the fusion peptides described herein. Variations of these sequences are also useful for imaging one or more internal body structures, detecting one or more rare earth elements in a biosample from a subject, detecting one or more rare earth elements in a sample (e.g., an environmental sample), and/or removing one or more rare earth elements from a sample (e.g., an environmental sample). For example, peptides having at least 70% sequence identity (i.e., at least 75% sequence identity, at least 80% sequence identity, at least 85% sequence identity, at least 90% sequence identity, at least 95% sequence identity, at least 96% sequence identity, at least 97% sequence identity, at least 98% sequence identity, or at least 99% sequence identity) to alternatively SEQ ID NOs:6, 7, and 10-13 are also useful for the described purposes, provided the peptides retain—broadly—the capacity to bind one or more rare earth elements and display an enhanced fluorescence following exposure to an excitation wavelength.

The fusion peptides described herein may be incorporated in a composition, such as a composition for detecting a rare earth element in a subject, biosample, and/or an environmental sample. The composition may detect a lanthanide, an actinide, scandium, yttrium, and combinations thereof. In a specific embodiment, the composition detects a rare earth element selected from the group consisting of scandium, yttrium, lanthanum, cerium, praseodymium, neodymium, promethium, samarium, europium, gadolinium, terbium, dysprosium, holmium, erbium, thulium, ytterbium, lutetium, thorium, protactinium, uranium, neptunium, plutonium, americium, curium, berkelium, californium, einsteinium, fermium, mendelevium, nobelium, lawrencium, and combinations thereof. In a specific embodiment, the composition detects gadolinium. In a particular embodiment, the composition detects gadolinium (III).

The composition may vary according to how the fusion peptide is to be used. In an embodiment, the fusion peptide may be in a solution, such as a buffered solution. The buffered solution may further comprise enzymes and/or metabolites. In some embodiments, the buffered solution may further comprise a biosample. In other embodiments, the buffered solution may further comprise an environmental sample. In other embodiments, the fusion peptide may be in a powder, such as lyophilized powder or a vacuum powder. Alternatively, the fusion peptide may be formulated for use in a column. In an embodiment, the fusion peptide may be suspended or bound to a resin, synthetic polymer, biopolymer, or hydrogel biodegradable polymer. In yet another embodiment, the fusion peptide may be conjugated, affixed, adsorbed, or otherwise attached to a surface. In such embodiments, the surface may be selected from the group consisting of a membrane, filter, dipstick, sample stick, paper, and film.

NUCLEOTIDES AND VECTORS: Contemporary molecular biologists know how to make nucleic acids that express the fusion peptides described herein, and how to express such nucleic acids in cells to obtain the relevant proteins. Further embodiments provided herein include nucleic acids or polynucleotides that encode the fusion peptides. For example, nucleic acids of SEQ ID NOs:8 and 9 encode fusion peptides described herein. The ordinary molecular biologist knows how to alter the nucleotide sequence of SEQ ID NOs:8, 9, or 10-13 to encode fusion peptide of SEQ ID NOs:6, 7, and 21-24, respectively, and appropriate variants thereof (e.g., variants having at least 70% identity (e.g., at least 75%, at least 80%, at least 85%, at least 90%, at least 95%, at least 96%, at least 97%, at least 98%, or at least 99% to any one of SEQ ID NOs:6 and 7)). Non-limiting examples of nucleic acids encoding the peptide chains of SEQ ID NOs:6, 7, and 10-13 are provided herein as SEQ ID NOs:8, 9 and 21-24, respectively.

Additionally or alternatively, the nucleic acids described herein can be incorporated into a vector (e.g., a transfection vector or a viral transduction vector). Such vectors can then be transformed, transfected, or transduced (as appropriate) into bacteria (e.g., E. coli), mammalian cells (e.g., CHO cells, mouse myeloma lymphoblastoid cells, and human embryonic kidney cells (e.g., HEK-293 cells)), yeast (e.g., S. cerevisiae or P. pastoris cells), and insect cells (e.g., Sf9 cells, Sf21 cells, and Hi-5 cells)) followed by culturing the bacteria or eukaryotic cells to produce the fusion peptides. Bacteria or cells transformed, transfected, or transduced with vectors comprising a fusion peptide can be cultured on a microscale in volumes measured in milliliters or cultured on an industrial scale in one or more bioreactors and/or all productions scales in between. The scale and nature of the culturing conditions are not particularly limited and are at the discretion of the artisan. Where industrial scale culturing occurs, the protein may be synthesized in one bioreactor and isolated from the cultured cells in a second or different bioreactor.

In some embodiments, the nucleic acid encoding the fusion peptide is incorporated into a cell. Such cells may translate the nucleic acid encoding the fusion peptide to express the fusion peptide. The cells may be bacterial cells, mammalian cells, yeast cells, and insect cells. In some embodiments, the cell comprises the fusion peptide comprising a sequence having at least 70% sequence identity to SEQ ID NO:6. In a particular embodiment, the cell comprises SEQ ID NO:6. In another embodiment, the cell comprises the fusion peptide comprising a sequence having at least 70% sequence identity to SEQ ID NO:7. In a particular embodiment, the cell comprises SEQ ID NO:7. In some embodiments, the cell comprises the fusion peptide comprising a sequence having at least 70% sequence identity to SEQ ID NO:10. In a particular embodiment, the cell comprises SEQ ID NO:10. In some embodiments, the cell comprises the fusion peptide comprising a sequence having at least 70% sequence identity to SEQ ID NO:11. In a particular embodiment, the cell comprises SEQ ID NO:11. In some embodiments, the cell comprises the fusion peptide comprising a sequence having at least 70% sequence identity to SEQ ID NO:12. In a particular embodiment, the cell comprises SEQ ID NO:12. In some embodiments, the cell comprises the fusion peptide comprising a sequence having at least 70% sequence identity to SEQ ID NO:13. In a particular embodiment, the cell comprises SEQ ID NO:13.

METHODS OF USE: The fusion peptides and compositions thereof described herein can be used in a variety of ways. In one embodiment is a method for imaging one or more internal body structures in a subject comprising the steps of: a. administering to the subject a contrast agent; b. administering to the subject the composition comprising the fusion peptide; c. detecting an amount of fluorescence of the fusion peptide; and d. preparing an image of the one or more internal body structures based on the location and intensity of the fluorescence. Examples of devices that can induce and detect fluorescence can be found in U.S. Pat. No. 10,517,483 and US 2020/0104998, both of which are incorporated herein by reference. In a specific embodiment, the contrast agent comprises gadolinium, particularly gadolinium (III). In a further embodiment, the subject has or is suspected of having damage to one or more internal body structures.

In another embodiment is a method for detecting one or more rare earth elements in a biosample from a subject having ingested, suspected of having ingested, and/or otherwise been exposed to a rare earth element, the method comprising the steps of: a. obtaining the biosample from the subject; b. contacting the biosample with the composition comprising the fusion peptide; c. exposing the biosample to light; and d. determining the amount of fluorescence of the biosample. In a further embodiment, the rare earth element is selected from the group consisting of a lanthanide, an actinide, scandium, yttrium, and combinations thereof. In a still further embodiment, the one or more rare earth elements is selected from the group consisting of scandium, yttrium, lanthanum, cerium, praseodymium, neodymium, promethium, samarium, europium, gadolinium, terbium, dysprosium, holmium, erbium, thulium, ytterbium, lutetium, thorium, protactinium, uranium, neptunium, plutonium, americium, curium, berkelium, californium, einsteinium, fermium, mendelevium, nobelium, lawrencium, and combinations thereof. In a specific embodiment, the rare earth element includes gadolinium, particularly gadolinium (III). In certain embodiments, the biosample is selected from the group consisting of blood, serum, plasma, mucus, saliva, skin, urine, a biopsy, resected tumor, tissue, organ, and combinations thereof.

In an embodiment is a method for detecting one or more rare earth elements in a sample comprising or suspected of comprising a rare earth element, the method comprising the steps of: a. obtaining the sample; b. contacting the sample with the composition comprising the fusion peptide; c. exposing the sample to light; and d. determining the amount of fluorescence of the sample. In a further embodiment, the rare earth element is selected from the group consisting of a lanthanide, an actinide, scandium, yttrium, and combinations thereof. In a still further embodiment, the one or more rare earth elements is selected from the group consisting of scandium, yttrium, lanthanum, cerium, praseodymium, neodymium, promethium, samarium, europium, gadolinium, terbium, dysprosium, holmium, erbium, thulium, ytterbium, lutetium, thorium, protactinium, uranium, neptunium, plutonium, americium, curium, berkelium, californium, einsteinium, fermium, mendelevium, nobelium, lawrencium, and combinations thereof. In a specific embodiment, the rare earth element includes gadolinium, particularly gadolinium (III). In a still further embodiment, the method further comprises the step of quantifying the amount of one or more rare earth elements. In a specific embodiment, the amount of the one or more rare earth elements is determined by correlating the amount of fluorescence of the sample to a quantity of the one or more rare earth elements. In some embodiments, the sample is an environmental sample, such as a sample selected from the group consisting of soil, river, lake, beach, coastal water, waste water, drinking water, a plant, algae, a fish, a crustacean, ore, gangue, rock, sewage, landfill, and combinations thereof. The source of the light is not particularly limited.

In an embodiment is a method for removing one or more rare earth elements from a site containing or suspected of containing the one or more rare earth elements, the method comprising: a. obtaining a sample from the site; b. contacting the sample with the composition comprising the fusion peptide; c. incubating the sample for a period of time for the fusion peptide to bind to the one or more rare earth elements; d. isolating and removing the fusion peptide from the sample; and e. returning the sample of step d to the site. In some embodiments, steps a.- e. are repeated two or more times or until the site is free or substantially free of the one or more rare earth elements. In a still further embodiment, the method further comprises the step of determining the amount of one or more rare earth elements in the sample, wherein said step is performed between steps c. and d. In a still further embodiment, the amount of the one or more rare earth elements is determined by exposing the sample to light, determining the amount of fluorescence of the sample, and correlating the amount of fluorescence of the sample to a quantity of the one or more rare earth elements. The source of the light is not particularly limited. In some embodiments, the site is an environmental site, such as an environmental site selected from the group consisting of soil, river, lake, beach, coastal water, waste water, drinking water, sewage, landfill, and combinations thereof.

### EXAMPLES

The following example is provided to further illustrate the fusion peptide disclosed herein but should not be construed as in any way limiting its scope.

MANUFACTURING: The fusion peptides described herein can be produced by inducing expression of the fusion peptide bacteria (e.g., E. coli), mammalian cells (e.g., CHO cells, mouse myeloma lymphoblastoid cells, and human embryonic kidney cells (e.g., HEK-293 cells)), yeast (e.g., S. cerevisiae or P. pastoris cells), and/or insect cells (e.g., Sf9 cells, Sf21 cells, and Hi-5 cells)) followed by isolation of the fusion peptide.

By way of a non-limiting example, a vector containing a nucleotide sequence of SEQ ID NO:8 or SEQ ID NO:9 is placed in the vector pET 101. E. coli was transformed with the vector according to standard transformation protocols. The E. coli was cultured at 30° C. for 24 hours. Following the culturing, the E. coli was harvested and lysed. The fusion peptides according to SEQ ID NO:6 (translated from SEQ ID NO:8; also known as GLamouR 1.0) or SEQ ID NO:7 (translated from SEQ ID NO:9; also known as GLamouR 2.2) were isolated and purified via the HIS sequence in the tail domain by cobalt resin purification. The harvested proteins were utilized in various assays.

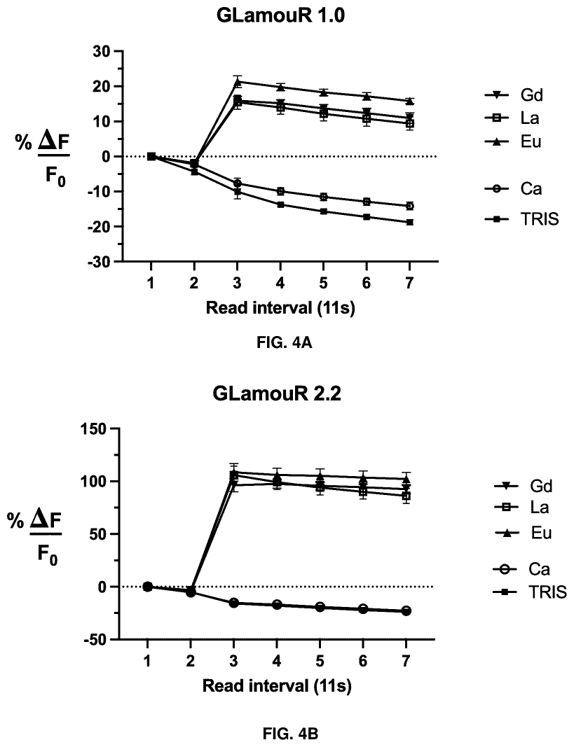

RARE EARTH ELEMENT-INDUCED FLUORESCENCE OF THE FUSION PEPTIDES: SEQ ID NO:6 (GLamouR 1.0) and SEQ ID NO:7 (GLamouR 2.2) were incubated with calcium, europium, gadolinium, lanthanum, or TRIS buffer (as a negative control). Following incubation, samples were exposed to light (488 nm) and fluorescence (%ΔF/Fₒ) at 510 nm was measured. As shown in FIGS. 4A and 4B, neither SEQ ID NO:6 nor SEQ ID NO:7 fluoresced following incubation with calcium or TRIS. However, as shown in FIGS. 4A and 4B, both fusion peptides displayed increased fluorescence following incubation with each of the tested rare earth elements (Eu, Gd, and La). SEQ ID NO:7, which comprises a chimera of lanmodulin and calmodulin in the rare earth element detection domain, showed almost a 100% increase in fluorescence following incubation with a rare earth element.

The above data demonstrates the desirable properties of the fusion peptide described herein. The fusion peptides are sensitive and specific for numerous rare earth elements and able to detect as little as 100 nM (approximately 14 ppb) amounts of rare earth elements such as lanthanum. Further, the fusion peptides described herein have a short activation time (approximately 22 seconds after contact) and a short relaxation time (approximately 220 milliseconds). Thus, the fusion peptides described herein provide fast, reliable results to detect and quantify the amount of rare earth element in a sample.

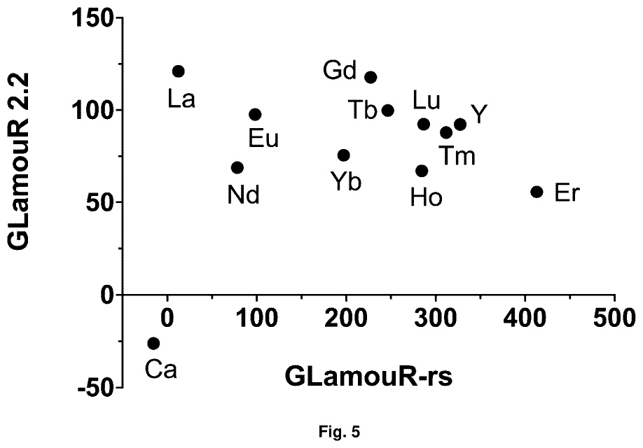

BINDING OF VARIOUS RARE EARTH ELEMENTS: The capacity of SEQ ID NO:7 (GLamouR 2.2) and red-shifted SEQ ID NO:12 (GLamouR-rs) to bind to various rare earth elements was analyzed. Wells of a 96-well plate were filled with 240 μL of 25 mM TRIS buffer, pH 7.4. 5 μL of a solution containing 10 nM SEQ ID NO:7 or 10 μM SEQ ID NO:12 were added to each well. After mixing, the samples were exposed to light (488 nm) and fluorescence was measured at 510 nm to establish a baseline fluorescence. 5 μL of calcium (control) and various rare earth elements (yttrium, lanthanum, neodymium, europium, gadolinium, terbium, holmium, erbium, thulium, ytterbium, and lutetium) at a concentration of 100 μM were individually added to the appropriate wells. Following shaking for 3 minutes, the samples were exposed to light (488 nm) and the fluorescence at 510 nm was measured for each sample. FIG. 5 shows the fluorescence of SEQ ID NO:7 (GLamouR 2.2) and red-shifted SEQ ID NO:12 (GLamouR-rs). As shown in the figure, the fusion peptides described herein are capable of detecting numerous rare earth elements. Further, the data demonstrate that the fusion peptides can be modified for high sensitivity to particular rare earth elements.

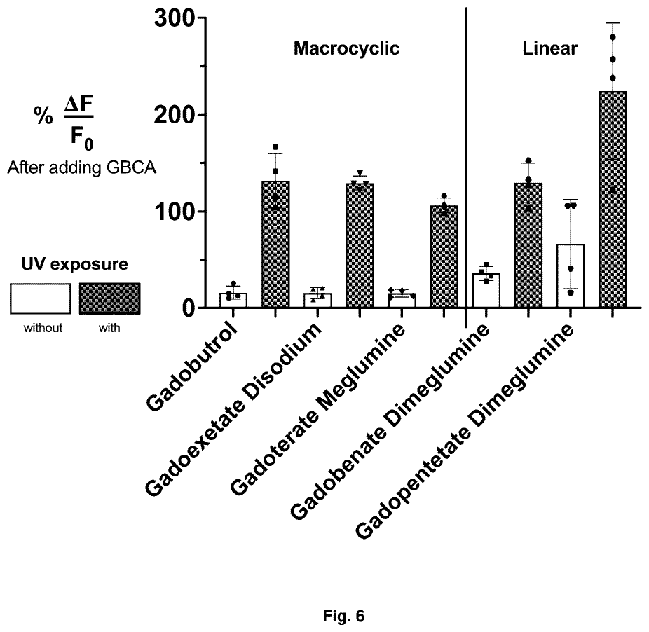

BINDING OF VARIOUS GADOLINIUM-BASED CONTRAST AGENTS: The capacity of SEQ ID NO:7 (GLamouR 2.2) to bind to various gadolinium-based contrast agents (GBCAs) was analyzed. Wells of a 96-well plate were filled with 240 μL of 25 mM TRIS buffer, pH 7.4. 5 μL of a solution containing 10 nM SEQ ID NO:7 was added to each well. After mixing, the samples were exposed to light (488 nm) and fluorescence was measured at 510 nm to establish a baseline fluorescence. 5 μL of various GBCAs (gadobutrol, gadoexetate disodium, gadoterate meglumine, gadobenate dimeglumine, and gadopentetate dimeglumine) at a concentration of 10 mM were individually added to the appropriate wells. Following shaking for 3 minutes, the samples were exposed to light (488 nm) and the fluorescence at 510 nm was measured for each sample. FIG. 6 shows the fluorescence of SEQ ID NO:7 (GLamouR 2.2) in the absence (grey bars) or presence (hatch bars) of UV exposure. As shown in the figure, the fusion peptides described herein are capable of detecting numerous GBCAs regardless of the GBCA configuration (e.g., linear or macrocyclic). Further, the data demonstrate that the rare earth elements used in MRIs and excreted from the patient will break free from their chelates. The fusion peptides described herein are capable of both detecting the rare earth elements as well as scavenging rare earth elements from the environment.

The use of the terms “a” and “an” and “the” and “at least one” and similar referents in the context of describing the fusion peptide and related uses (especially in the context of the following claims) are to be construed to cover both the singular and the plural, unless otherwise indicated herein or clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Particular embodiments of the fusion peptide are described herein, including the best mode known to the inventors for carrying out the invention. Variations of those particular embodiments may become apparent to those of ordinary skill in the art upon reading the foregoing description. The inventors expect skilled artisans to employ such variations as appropriate, and the inventors intend for the fusion peptide to be practiced otherwise than as specifically described herein. Accordingly, the fusion peptide described herein includes all modifications and equivalents of the subject matter recited in the claims appended hereto as permitted by applicable law. Moreover, any combination of the above-described elements in all possible variations thereof is encompassed by the described fusion peptide unless otherwise indicated herein or otherwise clearly contradicted by context.

What is claimed is:

1. A fusion peptide comprising a single fluorescent domain and a rare earth element detection domain.

2. The fusion peptide according to claim 1, wherein the fluorescent domain comprises an amino acid sequence having at least 85% sequence identity to a fluorescent protein selected from the group consisting of enhanced green fluorescent protein (EGFP), circular permutated EGFP (cpEGFP), green fluorescent protein (GFP), enhanced yellow fluorescent protein (EYFP), enhanced blue fluorescent protein (EBFP), enhanced cyan fluorescent protein (ECFP), enhanced red fluorescent protein (ERFP), enhanced orange fluorescent protein (EOFP) and mApple.

3. The fusion peptide according to claim 2, wherein the fluorescent domain comprises an amino acid sequence having at least 85% sequence identity to SEQ ID NO:1.

4. The fusion peptide according to claim 2, wherein the fluorescent domain comprises two or more pieces of the fluorescent protein separated by an amino acid sequence.

5. The fusion peptide according to claim 5, wherein the fluorescent domain comprises an amino acid sequence having at least 85% sequence identity to an amino acid sequence selected from the group consisting of SEQ ID NO:14, SEQ ID NO:15, SEQ ID NO:16, or a combination of SEQ ID NOs:2 and 3.

6. The fusion peptide according to claim 1, wherein the rare earth element detection domain comprises a protein selected from the group consisting of lanmodulin and a chimera of lanmodulin and calmodulin.

7. The fusion peptide according to claim 6, wherein the rare earth element detection domain comprises an amino acid sequence having at least 85% sequence identity to an amino acid sequence selected from the group consisting of SEQ ID NO:4, SEQ ID NO:5, SEQ ID NO:17, SEQ ID NO:18, SEQ ID NO:19, and SEQ ID NO:20.

8. The fusion peptide according to claim 1, further comprising a leader domain comprising an amino acid sequence having at least 70% sequence identity to M13 or RS20, wherein the leader domain is conjugated to the fluorescent domain.

9. The fusion peptide according to claim 8, further comprising a tail domain, wherein the tail domain comprises an amino acid sequence having at least 70% sequence identity to an amino acid sequence selected from the group consisting of V5, HIS, ALFA, AviTag, C-tag, polyglutamine, polyarginine, E-tag, hemagglutinin, Myc, NE, Rho1D4, S-tag, SBP-tag, Softag, Spot-tag, Strep-tag, T7, TC-tag, Ty-tag, VSV, and Xpress.

10. The fusion peptide of claim 1, wherein the fluorescent domain comprises cpEGFP or mApple and the rare earth element detection domain comprises a chimera of lanmodulin and calmodulin.

11. The fusion peptide according to claim 1, wherein the fusion peptide comprises an amino acid sequence having at least 85% sequence homology to an amino acid sequence selected from the group consisting of SEQ ID NO: 6, SEQ ID NO: 7, SEQ ID NO: 10, SEQ ID NO: 11, SEQ ID NO: 12, and SEQ ID NO: 13.

12. The fusion peptide according to claim 11, wherein the fusion peptide comprises an amino acid sequence selected from the group consisting of SEQ ID NO: 6, SEQ ID NO: 7, SEQ ID NO: 10, SEQ ID NO: 11, SEQ ID NO: 12, and SEQ ID NO: 13.

13. A fusion peptide comprising a fluorescent domain and a rare earth element detection domain, wherein the fluorescent domain comprises two or more pieces of a fluorescent protein separated by an amino acid sequence.

14. The fusion peptide according to claim 13, wherein the fluorescent domain comprises an amino acid sequence having at least 85% sequence identity to a fluorescent protein selected from the group consisting of enhanced green fluorescent protein (EGFP), circular permutated EGFP (cpEGFP), green fluorescent protein (GFP), enhanced yellow fluorescent protein (EYFP), enhanced blue fluorescent protein (EBFP), enhanced cyan fluorescent protein (ECFP), enhanced red fluorescent protein (ERFP), enhanced orange fluorescent protein (EOFP) and mApple.

15. The fusion peptide according to claim 14, wherein the fluorescent domain comprises an amino acid sequence having at least 85% sequence identity to an amino acid sequence selected from the group consisting of SEQ ID NO:14, SEQ ID NO:15, SEQ ID NO:16, or a combination of SEQ ID NOs:2 and 3.

16. The fusion peptide according to claim 13, wherein the rare earth element detection domain comprises a protein selected from the group consisting of lanmodulin and a chimera of lanmodulin and calmodulin.

17. The fusion peptide according to claim 16, wherein the rare earth element detection domain comprises an amino acid sequence having at least 85% sequence identity to an amino acid sequence selected from the group consisting of SEQ ID NO:4, SEQ ID NO:5, SEQ ID NO:17, SEQ ID NO:18, SEQ ID NO:19, and SEQ ID NO:20.

18. The fusion peptide according to claim 13, further comprising a leader domain comprising an amino acid sequence having at least 70% sequence identity to M13 or RS20, wherein the leader domain is conjugated to the fluorescent domain.

19. The fusion peptide according to claim 18, further comprising a tail domain, wherein the tail domain comprises an amino acid sequence having at least 70% sequence identity to an amino acid sequence selected from the group consisting of V5, HIS, ALFA, AviTag, C-tag, polyglutamine, polyarginine, E-tag, hemagglutinin, Myc, NE, Rho1D4, S-tag, SBP-tag, Softag, Spot-tag, Strep-tag, T7, TC-tag, Ty-tag, VSV, and Xpress.

20. The fusion peptide of claim 19, wherein the fluorescent domain comprises cpEGFP or mApple and the rare earth element detection domain comprises a chimera of lanmodulin and calmodulin.

21. The fusion peptide according to claim 13, wherein the fusion peptide comprises an amino acid sequence having at least 85% sequence homology to an amino acid sequence selected from the group consisting of SEQ ID NO: 6, SEQ ID NO: 7, SEQ ID NO: 10, SEQ ID NO: 11, SEQ ID NO: 12, and SEQ ID NO: 13.

22. The fusion peptide according to claim 21, wherein the fusion peptide comprises an amino acid sequence selected from the group consisting of SEQ ID NO: 6, SEQ ID NO: 7, SEQ ID NO: 10, SEQ ID NO: 11, SEQ ID NO: 12, and SEQ ID NO: 13.

23. A composition for detecting a rare earth element comprising a fusion peptide comprising a single fluorescent domain and a rare earth element detection domain.

24. The composition according to claim 23, wherein the fluorescent domain comprises an amino acid sequence having at least 85% sequence identity to a fluorescent protein selected from the group consisting of enhanced green fluorescent protein (EGFP), circular permutated EGFP (cpEGFP), green fluorescent protein (GFP), enhanced yellow fluorescent protein (EYFP), enhanced blue fluorescent protein (EBFP), enhanced cyan fluorescent protein (ECFP), enhanced red fluorescent protein (ERFP), enhanced orange fluorescent protein (EOFP) and mApple.

25. The composition according to claim 24, wherein the fluorescent domain comprises two or more pieces of the fluorescent protein separated by an amino acid sequence.

26. The composition according to claim 23, wherein the rare earth element detection domain comprises a protein selected from the group consisting of lanmodulin and a chimera of lanmodulin and calmodulin.

27. The composition according to claim 23, wherein the fusion peptide comprises an amino acid sequence having at least 85% sequence homology to an amino acid sequence selected from the group consisting of SEQ ID NO: 6, SEQ ID NO: 7, SEQ ID NO: 10, SEQ ID NO: 11, SEQ ID NO: 12, and SEQ ID NO: 13.

28. The composition according to claim 23, wherein the fusion peptide comprises an amino acid sequence selected from the group consisting of SEQ ID NO: 6, SEQ ID NO: 7, SEQ ID NO: 10, SEQ ID NO: 11, SEQ ID NO: 12, and SEQ ID NO: 13.

29. A nucleic acid encoding the fusion peptide comprising a fluorescent domain and a rare earth element domain according to claim 1.

30. A vector comprising the nucleic acid according to claim 29.
